(19) United States
(12) Patent Application Publication
(10) Pub. No.: US 2007/0010749 A1
(43) Pub. Date: Jan. 11, 2007

(54) Diagnostic device and the method using the same

(75) Inventor: Chao-Peng Meng, Taipei (TW)

Correspondence Address: BIRCH STEWART KOLASCH & BIRCH, PO BOX 747, FALLS CHURCH, VA, 22040-0747, US

(73) Assignee: CARDIO VASCULAR METRICS INC.

(21) Appl. No.: 11/414,236

(22) Filed: May 1, 2006

(30) Foreign Application Priority Data
Jul. 6, 2005 (TW) 094122837

(51) Int. Cl. A61B 5/02

(52) U.S. Cl. 600/490; 600/485; 600/500

(57) ABSTRACT

A diagnostic device for measuring pulse waves from an upper arm is provided. The device includes a cuff, a sensor, and a processing circuit. The cuff provides continuously a pressure to arteries of the upper arm. Then the sensor measures a plurality of pulse waves from an upper arm in response to the pressure. The plurality of pulse waves includes a single pulse wave. The processing circuit, coupled to the sensor, generates data of the plurality of pulse waves. According to data of the single pulse wave extracted from the data of the plurality of pulse waves, the processing circuit computes and outputs diagnostic data.

### CROSS REFERENCE TO RELATED APPLICATIONS

This application claims the right of priority based on Taiwan Patent Application No. 094122837 entitled “Diagnostic Device and the Method using the Same”, filed on Jul. 6, 2005, which is incorporated herein by reference and assigned to the assignee herein.

### FIELD OF INVENTION

The present invention relates to a diagnostic device and the method using the same. Particularly, the present invention relates to a diagnostic device and the method using the same in the Chinese medical diagnosis.

### BACKGROUND OF THE INVENTION

Typical Chinese medicine practitioners rely on “pulse taking” from the writst to diagnose the patients. However, no practical or reliable diagnostic device has been provided for taking and analyzing the pulses automatically and statistically. The prior-art device utilized a tonometry to detect the reaction of arteries of the wrist under varying external pressures, but the operation was complicated and the result was not accurate enough.

On the other hand, in the modern Western medicine, the techniques to measure the blood pressure are quite mature. Typically, a cuff is bound around the upper arm to sense the blood pressure of arteries. For example, the DP200M of Pulse Metric, Inc. measures the blood pressure automatically, as well as the pulse rate and other data of the pulse. Furthermore, it produces a graphical diagram to present the measured data.

In the Chinese medicine theory, “pulse taking” is not limited to performing on the wrist. Other body positions can also be adopted. And the “pulse taking” of Chinese medicine is similar to the blood pressure measurement of Western medicine. Both methodologies press the vessel by an external pressure, and then lower the external pressure gradually and detect the reaction of the vessel. In Chinese medicine, the fingers apply the external pressure, and then the fingers also detect the reaction of the vessel. Therefore, to obtain more accurate diagnostic result, it will be advantageous to apply the blood pressure measurement of Western medicine in the “pulse taking” process of Chinese medicine.

### SUMMARY OF THE INVENTION

One aspect of the present invention is to provide a diagnostic device for measuring pulse waves and a method using the same.

Another aspect of the present invention is to provide an automatic diagnostic device for Chinese medicine and a method using the same.

Replacing a traditional Chinese medicine practitioner's three fingers touching the wrist of the patient, the present invention resides in that the cuff bound on the upper arm. That is, the present invention utilizes the cuff to provide different external pressures and then measures the corresponding pulse wave. Accordingly, the present invention obtains accurate and detailed diagnostic results.

In one embodiment, disclosed is a diagnostic device for measuring pulse waves from an upper arm. The device includes a cuff, a sensor, and a processing circuit. The cuff provides continuously a pressure to arteries of the upper arm. Then the sensor measures a plurality of pulse waves from an upper arm in response to the pressure. The plurality of pulse waves includes a single pulse wave. The processing circuit, coupled to the sensor, generates data of the plurality of pulse waves. According to data of the single pulse wave extracted from the data of the plurality of pulse waves, the processing circuit computes and outputs diagnostic data.

Also disclosed is a diagnostic method. The method includes: (a) providing a pressure continuously by a cuff to arteries of an upper arm during a measuring period; (b) measuring a plurality of pulse waves from the arteries in response to the pressure, and generating data of the plurality of pulse waves, the plurality of pulse waves comprising a single pulse wave; and (c) according to data of the single pulse wave extracted from the data of the plurality of pulse waves, computing diagnostic data.

The foregoing and other features of the invention will be apparent from the following more particular description of embodiment of the invention.

### BRIEF DESCRIPTION OF THE DRAWINGS

The present invention is illustrated by way of example and not intended to be limited by the figures of the accompanying drawing, in which like notations indicate similar elements.

FIG. 1 illustrates a diagnostic device according to an embodiment of the present invention;

FIGS. 2A-2C show the whole pulse wave diagrams according to the embodiments of the present invention;

FIGS. 2D-2E show the single pulse wave diagrams according to the embodiments of the present invention;

FIGS. 2F-2M show the diagnostic data according to the embodiments of the present invention;

FIG. 3 shows a selected single pulse wave diagram from the whole pulse wave diagram; the selected single pulse wave diagram is referred to as the systolic pulse wave diagram, the average pulse wave diagram, or the diastolic pulse wave diagram;

FIGS. 4A-4D show diagnostic data according to the embodiments of the present invention;

FIG. 5 illustrates a diagnostic system according to an embodiment of the present invention;

FIG. 6 illustrates a diagnostic system according to another embodiment of the present invention;

FIG. 7 is a flow chart of a diagnostic method according to an embodiment of the present invention.

### DETAILED DESCRIPTION

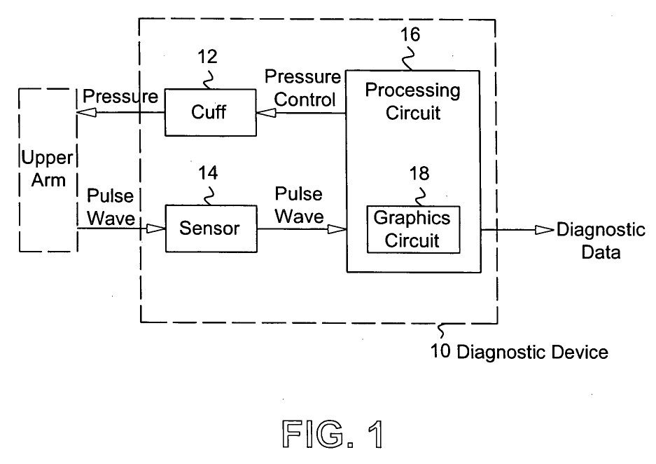

Referring to FIG. 1, the diagnostic device 10 includes a cuff 12, a sensor 14, and a processing 16. The cuff 12, bound on an upper arm, provides continuously a pressure to arteries of the upper arm. By gas filling or exhausting, the pressure provided by the cuff 12 can be varied. The pressure can be fixed, or time-varied, ie stepwise upward or downward. Those skilled in the art can refer to the cuff of the conventional electronic blood pressure meter to realize the cuff 12 of the present invention.

The sensor 14 measures a plurality of pulse waves from an upper arm in response to the pressure. The plurality of pulse waves include a single pulse wave. The plurality of pulse waves are preferably, but not limited to, continuous waves. The processing circuit 16, coupled to the sensor 14, generates data of the plurality of pulse waves. The processing circuit 16 can be a CPU for performing a specific software programs or an application specific integrated circuit (ASIC). In one embodiment, the processing circuit 16 includes the DP200M of Pulse Metric, Inc, to take the data of the plurality of pulse waves, which represent the pulse amplitudes over time. Furthermore, DP200M is able to detect a systolic pressure, an average pressure, or a diastolic pressure, in the way known to those skilled in the art. After the data of the plurality of pulse waves are obtained, the processing circuit 16 computes and outputs diagnostic data according to data of the single pulse wave extracted from the data of the plurality of pulse waves. In one embodiment, the processing circuit 16 performs the differential, integral, or averaging calculation for the extracted data. In another embodiment, the processing circuit 16 further includes a graphics circuit 18 for generating a pulse wave diagram based on the data of the single pulse wave or based on the data of the plurality of pulse waves. The graphics circuit 18 is connected to an output device (not shown), such as a monitor or a printer, to output the pulse wave diagram. Note that the graphics circuit 18 is optionally provided for showing diagrammatically the data of the pulse wave(s), and can be omitted in some embodiments.

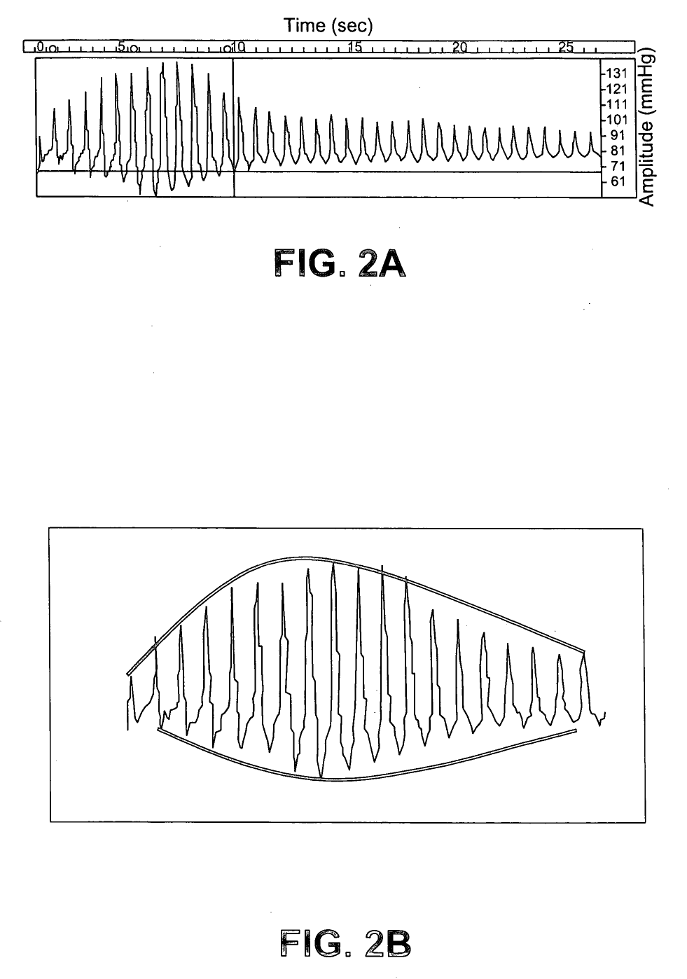

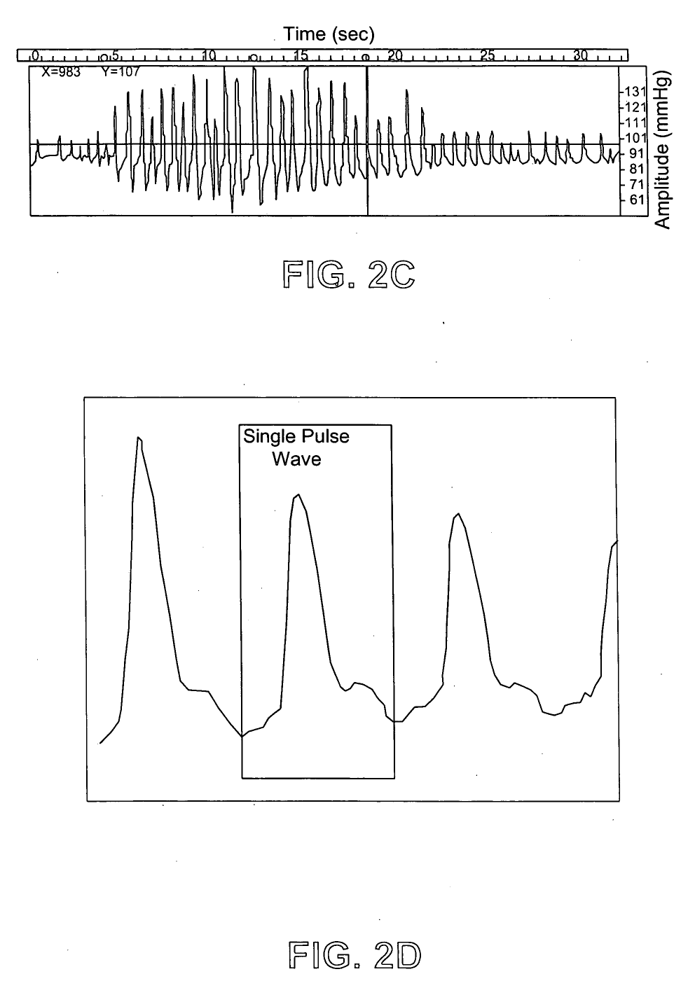

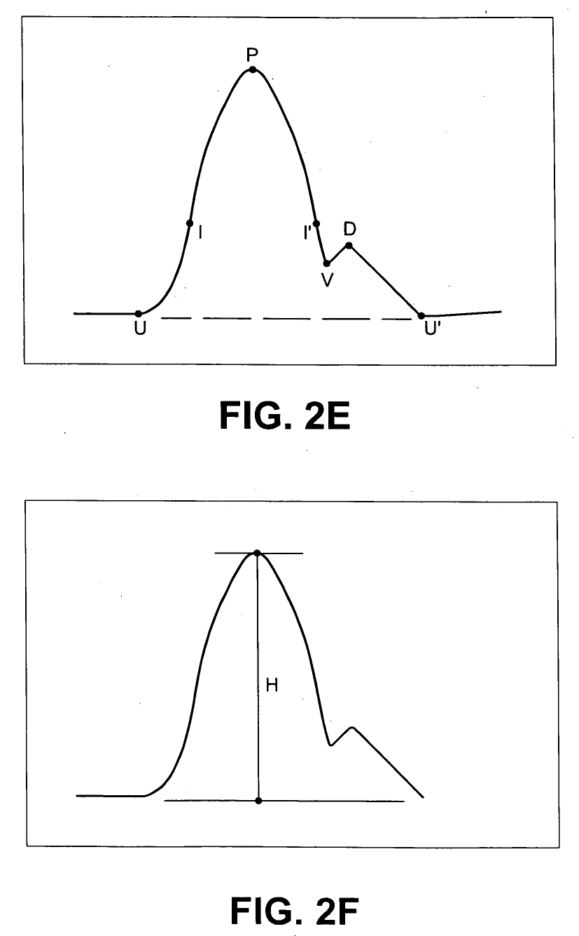

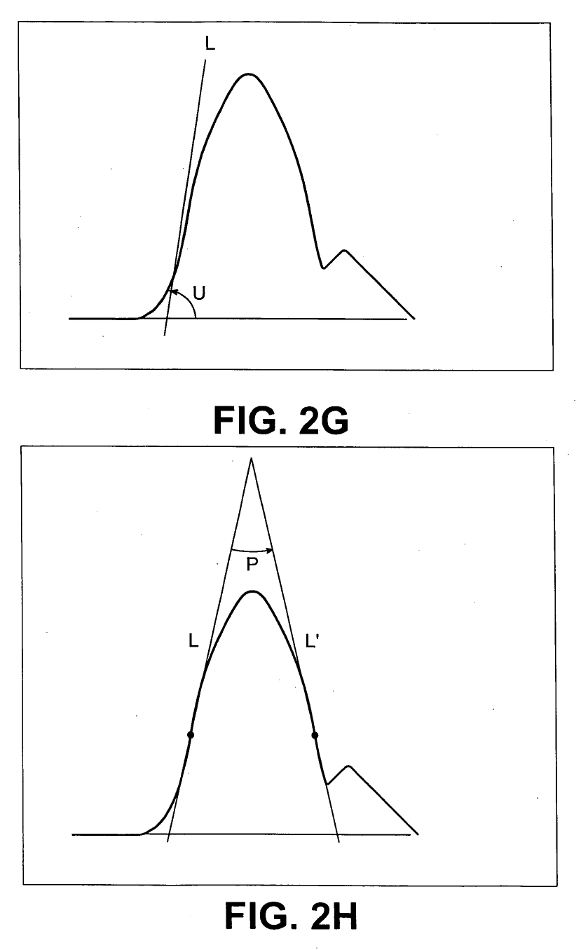

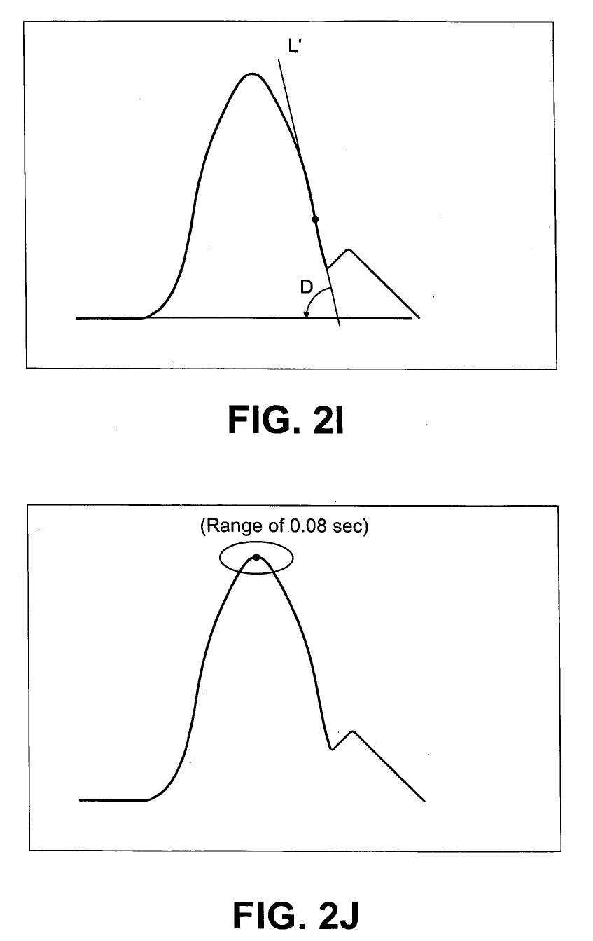

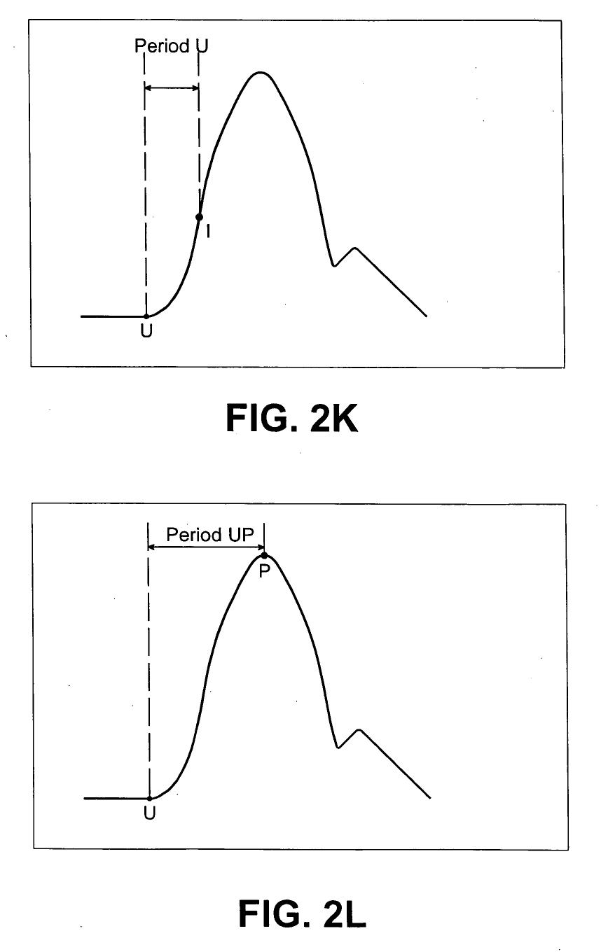

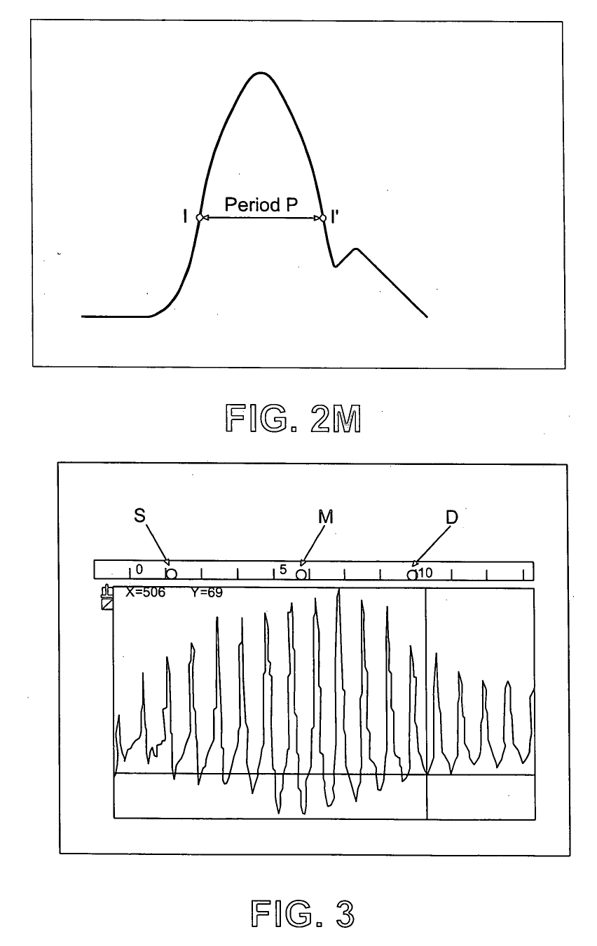

In the following FIGS. 2A-2M, FIG. 3, and FIGS. 4A-4D, the horizontal axis represents “Time (sec)”, and the vertical axis is referred to as “Amplitude (mmHg)”. FIG. 2A shows the whole pulse wave diagram generated by the graphics circuit 18 according to the data of the plurality of pulse waves. As exemplarily shown, under the varying pressures provided by the cuff, there are 37 single pulse waves during 28 seconds of the measuring period. Typically, as shown in FIG. 2B, the middle region of the whole pulse wave diagram is olive-shaped. However, as shown in FIG. 2C, the whole pulse wave diagram is different for a patient with the arrhythmia or the irregular pulses.

As shown in FIG. 2D, the single pulse wave begins from a wave trough to a next wave trough. Further, for the exemplary purpose, a typical single pulse wave is illustrated in FIG. 2E, wherein the U is the starting point, P is the apex point, UP is the upward branch, I is the most upward point (the maximum change of the amplitude over time on the upward branch UP), U′ is the ending point, PU′ is the downward branch, I′ is the most downward point (the maximum change of the amplitude over time on the downward branch PU′), and UU′ is the baseline. Also, the single pulse wave has a “Dichotic Notch”, VDU′, wherein V is the starting point of VDU′, and D is the apex point of VDU′.

As follows, the diagnostic data in some embodiments are defined in light of the description of FIG. 2E. Referring to FIG. 2F, “Pulse Height (H)” is measured from the apex point P to the baseline UU′. H is proportional to the “LV dp/dt Max (the maximum change of the pressure of the left ventricle over time)” and the “stroke volume”, but is inversely proportional to “SV compliance”.

Referring to FIG. 2G, “Angle U” is an angle between the baseline UU′ and a tangent line L to the most upward point I. Angle U is proportional to the “LV dp/dt Max”, “LV contractility”, “stroke volume”, and “BA compliance”, but is inversely proportional to “SV compliance”, “SV resistance”, and “BA resistance”.

Referring to FIG. 2H, “Angle P” is an angle between L and a tangent line L′ to the most downward point I′. Angle P is proportional to the “LV ejection time”, “SV compliance”, and “BA resistance”, but is inversely proportional to the “LV dp/dt Max”, “LV contractility”, “stroke volume”, and “BA compliance”.

Referring to FIG. 2I, “Angle D” is an angle between the baseline UU′ and L′.

Angle U is proportional to the “LV dp/dt Max”, “LV contractility”, and “stroke volume”, but is inversely proportional to “SV compliance”.

Referring to FIG. 2J, a top angle is delimited from the apex point P to two points respectively extending 0.04 sec range backward and forward from the apex point P. The sharpness of the top angle is proportional to “LV dp/dt Max”, “LV contractility”, “stroke volume”, and “BA compliance”, but is inversely proportional to the blood resistance.

Referring to FIG. 2K, “Period U” is the time starting from the point U to the point I. Period U is proportional to the “LV ejection time” and the blood resistance, but is inversely proportional to “LV contractility”, “stroke volume”, and “BA compliance”.

Referring to FIG. 2L, “Period UP” is the time starting from the point U to the point P. Period UP is proportional to the blood resistance, but is inversely proportional to “LV contractility”, “stroke volume”, and ““BA compliance”.

Referring to FIG. 2M, Period P is the time starting from the point I to the point I′. Period P is proportional to the blood resistance, but is inversely proportional to “LV contractility”, “stroke volume”, and ““BA compliance”.

As mentioned above, the processing circuit 16 can utilize the prior art device to further detect a systolic pressure, an average pressure, or a diastolic pressure. And the processing circuit 16 selects the data of the single pulse wave from the data of the plurality of pulse waves based on the systolic pressure, the average pressure, or the diastolic pressure. In one embodiment, the processing circuit 16 selects a single pulse wave diagram from the whole pulse wave diagram, according to the time when the pressure provided by the cuff 12 is equal to the systolic pressure, the average pressure, or the diastolic pressure, e.g., time S, M, or D shown in FIG. 3. Accordingly, the selected single pulse wave diagram is referred to as the systolic pulse wave diagram, the average pulse wave diagram, or the diastolic pulse wave diagram. In this embodiment, the first single pulse wave diagram after the time S is selected as the systolic pulse wave diagram, the first single pulse wave diagram after the time M is selected as the average pulse wave diagram, and the first single pulse wave diagram after the time D is selected as the diastolic pulse wave diagram. Those skilled in the art should appreciate that the systolic pulse wave diagram is referred to as the “deep pulse” in Chinese medicine, the average pulse wave diagram is referred to the as “middle pulse”, and the diastolic pulse wave diagram is referred to as the “floating pulse”. Accordingly, modern data processing and recording techniques can be introduced into Chinese Medicine for bringing a lot of benefits.

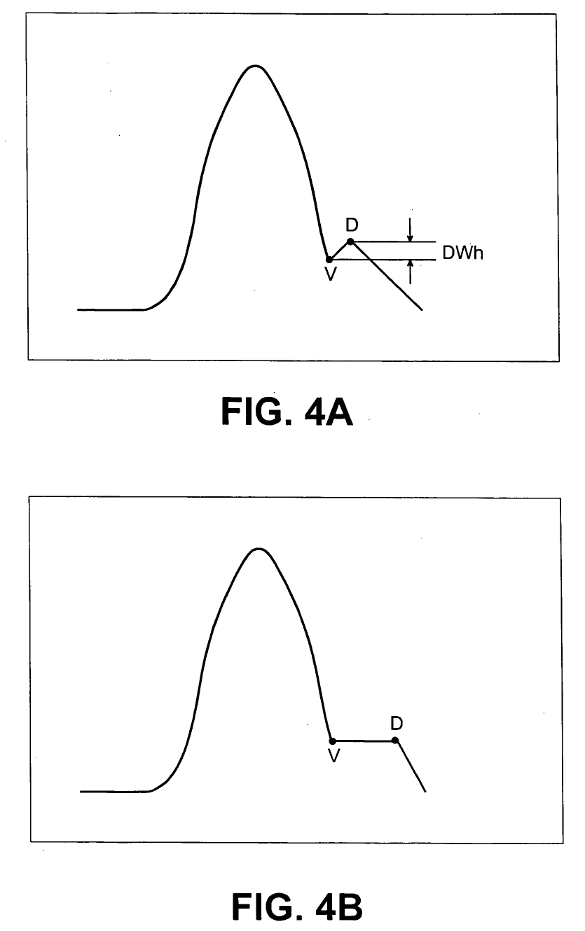

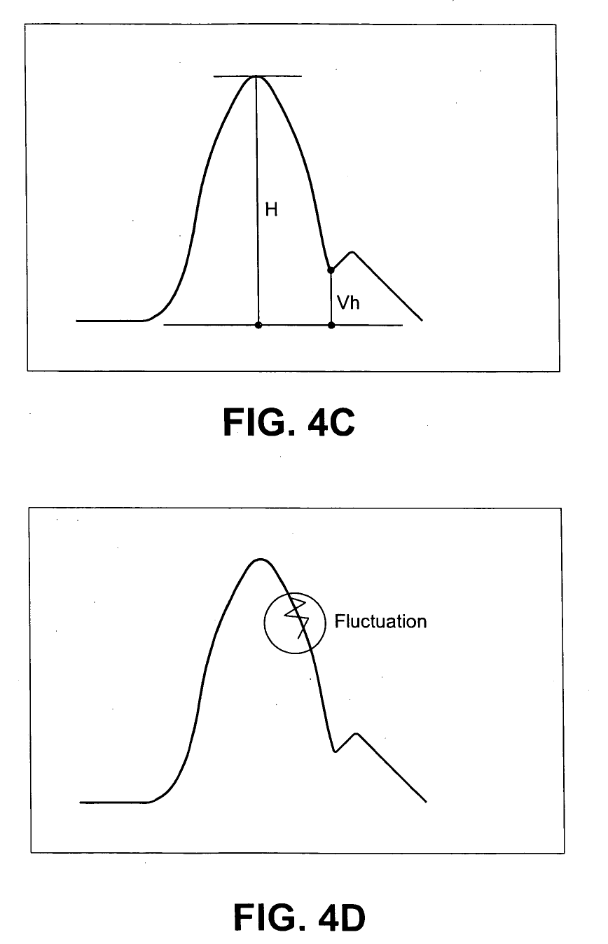

Typically, “Dicrotic Notch” usually appears after the time D, i.e., after the diastolic pulse wave. As shown in FIG. 4A, when the point D is noticeably higher than the point V, the processing circuit 16 computes the height (DWh) of “Dicrotic Notch” from the point D to the point V. Then as shown in FIG. 4B, when the point D is not higher than the point V, the processing circuit 16 computes the lasting interval of “Dicrotic Notch” from point b to the point V. Further referring to FIG. 4C, the starting height Vh of “Dicrotic Notch” is measured from the point V to the baseline UU′, and the processing circuit 16 computes the ratio of Vh to H. In one embodiment, after the time D, the processing circuit 16 selects several single pulse waves, computes and averages the DWhs, the lasting interval, or the ratios of Vh to H to output the diagnostic data. The average of DWh is proportional to “LV contractility”, “stroke volume”, and “BA compliance”, but is inversely proportional to the blood resistance. The average of the lasting interval is proportional to “LV ejection time” and “SV compliance”, but is inversely proportional to “LV dp/dt. Max”. The average of the ratios of Vh to H is proportional to to “LV ejection time” and “SV compliance”, but is inversely proportional to “LV contractility”. In addition to “Dicrotic Notch”, the pulse wave after the time D, i.e., after the diastolic pulse wave, may have a fluctuation between the point P and the point I′, as shown in FIG. 4D. The processing circuit 16 can compute the average amplitude of the fluctuation, which is inversely proportional to “LV dp/dt Max”, “LV contractility”, “stroke volume”, and “SV compliance”, but is proportional to the blood resistance.

Referring to FIG. 5, a diagnostic system 50 includes a server 52 and a diagnostic device 10. The server 52 stores an application program. The diagnostic device 10 is connected to the server 52 via a network 54. The network 54 can be the Internet or the local area network. The connection can be wired or wireless, direct or via other devices, such as a router. The diagnostic device 10 includes a cuff 12, a sensor 14, and a processing circuit 16. The cuff 12 provides continuously a pressure to arteries of the upper arm. The sensor 14 measures a plurality of pulse waves from the arteries of the upper arm in response to the pressure provided by the cuff 12. The plurality of pulse waves include a single pulse wave. The processing circuit 16, coupled to the sensor 14, generates data of the plurality of pulse waves. After the data of the plurality of pulse waves are obtained, the processing circuit 16 performs the application program downloaded from the server 52, to the compute the diagnostic data as shown FIGS. 2F-2M and 4A-4C, according to data of the single pulse wave extracted from the data of the plurality of pulse waves. The processing circuit 16 further includes a graphics circuit 18 for generating a pulse wave diagram based on the data of the single pulse wave or based on the data of the plurality of pulse waves. In one embodiment, the processing circuit 16 is included in a PC. Via a web browser program (e.g., Internet Explorer of Microsoft), the processing circuit 16 is connected to the server 52 to download the latest application program.

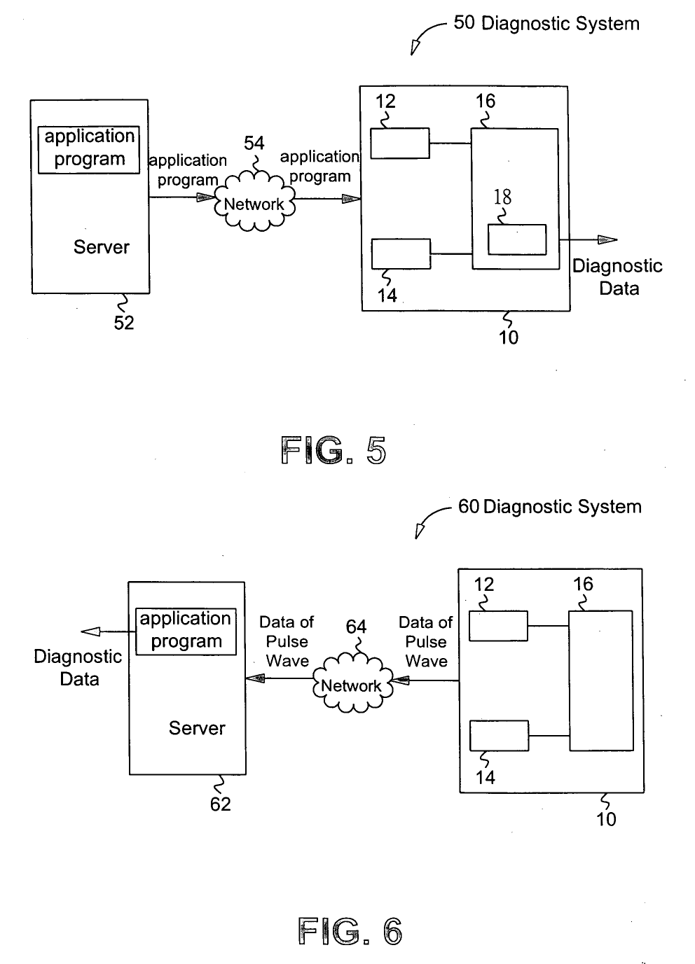

Referring to FIG. 6, a diagnostic system 60 includes a server 62, and a diagnostic device 10. The server 62 is provided for performing an application program. The diagnostic device 10 is connected to the server 62 via a network 64. The diagnostic device 10 includes a cuff 12, a sensor 14, and a processing circuit 16. The cuff 12 provides continuously a pressure to arteries of the upper arm. The sensor 14 measures a plurality of pulse waves from the arteries of the upper arm in response to the pressure provided by the cuff 12. The plurality of pulse waves include a single pulse wave. The processing circuit 16, coupled to the sensor 14, generates data of the plurality of pulse waves. Then the processing circuit 16 uploads the data of the plurality of pulse waves to the server 62. The server 62 performs the application program to the compute the diagnostic data as shown FIGS. 2F-2M and 4A-4C, according to data of the single pulse wave extracted from the data of the plurality of pulse waves. In one embodiment, the processing circuit 16 is included in a PC. Via a web browser program (e.g., Internet Explorer of Microsoft) or a FTP program, the processing circuit 16 is connected to the server 62 to upload the data of the plurality of pulse waves.

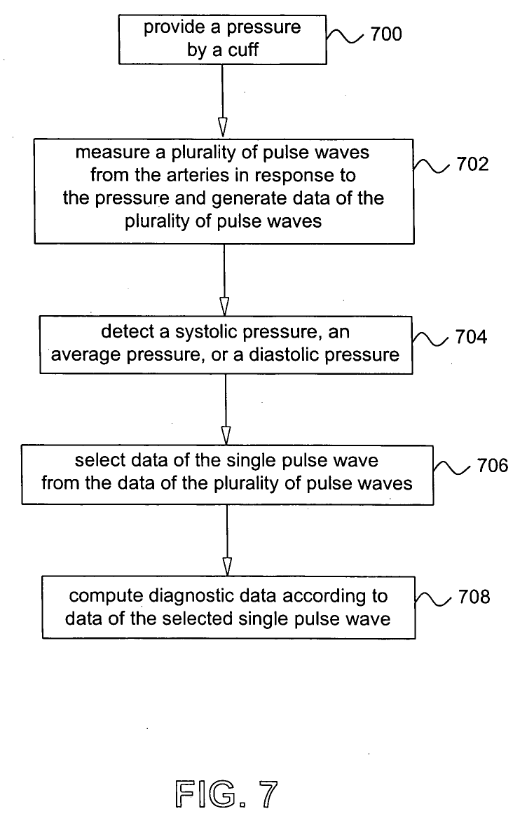

FIG. 7 illustrates a flow chart according to an embodiment of the present invention. At first, the step 700 is to provide a pressure continuously by a cuff to arteries of an upper arm during a measuring period. In one embodiment, the measuring period is 24 hours, so the patient is provided with a whole-day blood pressure monitor. Next, the step 702 is to measure a plurality of pulse waves from the arteries in response to the pressure and to generate data of the plurality of pulse waves. The plurality of pulse waves includes at least a single pulse wave. In one embodiment, the data of the plurality of pulse waves represent the pulse amplitudes over time. After that, the step 704 is to detect a systolic pressure, an average pressure, or a diastolic pressure. Then the step 706 is to select data of the single pulse wave from the data of the plurality of pulse waves, according to the time when the pressure provided by the cuff is equal to the systolic pressure, the average pressure, or the diastolic pressure, e.g., time S, M, or D shown in FIG. 3. Accordingly, the selected single pulse wave is referred to as the systolic pulse wave, the average pulse wave, or the diastolic pulse wave. Next, the step 708 is to compute diagnostic data according to data of the selected single pulse wave. In one embodiment, the step 702 is to further generate a pulse wave diagram based on the data of the single pulse wave (as shown in FIG. 2A), and the step 706 is to output the single pulse wave diagram.

The present invention also discloses a computer program including program code instructions for controlling the operation of a computer diagnostic device on which the program code executes, to perform the method illustrated in FIG. 7. Any suitable computer-readable storage medium may be utilized to store the computer program, including hard disks, CD-ROM, optical storage devices, magnetic storage devices, and/or the like.

While this invention has been described with reference to the illustrative embodiments, these descriptions should not be construed in a limiting sense. Various modifications of the illustrative embodiment, as well as other embodiments of the invention, will be apparent upon reference to these descriptions. It is therefore contemplated that the appended claims will cover any such modifications or embodiments as falling within the true scope of the invention and its legal equivalents.

1. A diagnostic device for measuring pulse waves from an upper arm, comprising:
a cuff for providing continuously a pressure to arteries of said upper arm,
a sensor for measuring a plurality of pulse waves from an upper arm in response to said pressure, said plurality of pulse waves comprising a single pulse wave; and
a processing circuit, coupled to said sensor, for generating data of said plurality of pulse waves,
wherein, according to data of said single pulse wave extracted from said data of said plurality of pulse waves, said processing circuit computes and outputs diagnostic data.

2. A diagnostic device according to claim 1, wherein said pressure is time varying.

3. A diagnostic device according to claim 1, wherein, further according said data of said plurality of pulse waves, said processing circuit computes said diagnostic data.

4. A diagnostic device according to claim 1, wherein said processing circuit detects a systolic pressure, an average pressure, or a diastolic pressure, and said processing circuit selects said data of said single pulse wave from said data of said plurality of pulse waves based on said systolic pressure, said average pressure, or said diastolic pressure.

5. A diagnostic device according to claim 1, wherein said processing circuit comprises a graphics circuit for generating a pulse wave diagram based on said data of said single pulse wave, and said processing circuit outputs said diagnostic data based on said pulse wave diagram.

6. A diagnostic device according to claim 5, wherein said diagnostic data can be Pulse Height, Angle U, Angle P, Angle D, Sharpness of a top angle, Period U, Period UP, or Period P of said single pulse wave.

7. A diagnostic device according to claim 6, wherein said processing circuit detects a systolic pressure, an average pressure, or a diastolic pressure, and said processing circuit selects said data of said single pulse wave from said data of said plurality of pulse waves based on said systolic pressure, said average pressure, or said diastolic pressure.

8. A diagnostic device according to claim 5, wherein said processing circuit detects a diastolic pressure, and said processing circuit selects said data of said single pulse wave from said data of said plurality of pulse waves based on said diastolic pressure;
wherein said single pulse wave includes a Dicrotic Notch, and said diagnostic data can be Height, lasting period, or starting height of said Dicrotic Notch.

9. A diagnostic system, comprising:
a computer server for storing an application program;
a diagnostic device for measuring pulse waves from an upper arm, said device being connected to said computer server via a network for downloading said application program, said diagnostic device comprising:
a cuff for providing continuously a pressure to arteries of said upper arm,
a sensor for measuring a plurality of pulse waves from an upper arm in response to said pressure, said plurality of pulse waves comprising a single pulse wave; and
a processing circuit, coupled to said sensor, for generating data of said plurality of pulse waves,
wherein, according to data of said single pulse wave extracted from said data of said plurality of pulse waves, said processing circuit performs said application program to compute and output diagnostic data.

10. A diagnostic system, comprising:
a computer server for performing an application program;
a diagnostic device for measuring pulse waves from an upper arm, said device being connected to said computer server via a network, said diagnostic device comprising:
a cuff for providing continuously a pressure to arteries of said upper arm,
a sensor for measuring a plurality of pulse waves from an upper arm in response to said pressure, said plurality of pulse waves comprising a single pulse wave; and
a processing circuit, coupled to said sensor, for generating data of said plurality of pulse waves,
wherein, according to data of said single pulse wave extracted from said data of said plurality of pulse waves, said computer server performs said application program to compute and output diagnostic data.

11. A diagnostic method, comprising:
(a) providing a pressure continuously by a cuff to arteries of an upper arm during a measuring period;
(b) measuring a plurality of pulse waves from said arteries in response to said pressure, and generating data of said plurality of pulse waves, said plurality of pulse waves comprising a single pulse wave; and
(c) according to data of said single pulse wave extracted from said data of said plurality of pulse waves, computing diagnostic data.

12. A diagnostic method according to claim 11, wherein said pressure is time varying.

13. A diagnostic method according to claim 11, wherein the step (c) further comprises: according said data of said plurality of pulse waves, said processing circuit computes said diagnostic data.

14. A diagnostic method according to claim 11, wherein the step (b) further comprises:
(b1) detecting a systolic pressure, an average pressure, or a diastolic pressure; and
(b2) selecting said data of said single pulse wave from said data of said plurality of pulse waves based on said systolic pressure, said average pressure, or said diastolic pressure.

15. A diagnostic method according to claim 11, wherein the step (c) comprises: generating a pulse wave diagram based on said data of said single pulse wave, and outputting said diagnostic data based on said pulse wave diagram.

16. A diagnostic method according to claim 15, wherein said diagnostic data can be Pulse Height, Angle U, Angle P, Angle D, Sharpness of a top angle, Period U, Period UP, or Period P of said single pulse wave.

17. A diagnostic method according to claim 16, where the step (b) further comprises:
(b1) detecting a systolic pressure, an average pressure, or a diastolic pressure; and
(b2) selecting said data of said single pulse wave from said data of said plurality of pulse waves based on said systolic pressure, said average pressure, or said diastolic pressure.

18. A diagnostic method according to claim 15, where the step (b) further comprises:
(b1) detecting a diastolic pressure; and
(b2) selecting said data of said single pulse wave from said data of said plurality of pulse waves based on said diastolic pressure;
wherein said single pulse wave includes a Dicrotic Notch, and said diagnostic data can be Height, lasting period, or starting height of said Dicrotic Notch.

19. A diagnostic method according to claim 11. said measuring period is 24 hours.

20. A computer program comprising program code instructions for controlling the operation of a computer diagnostic device on which the program code executes, to perform a method according to claim 11.
